(19) United States
(12) Patent Application Publication
(10) Pub. No.: US 2009/0248125 A1
(43) Pub. Date: Oct. 1, 2009

(54) INTEGRATED CONDUCTIVE PRESSURE SENSOR CAPSULE WITH CUSTOM MOLDED UNITARY OVERLAY

(75) Inventor: Thomas D. Brostrom, Wayzata, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/411,046

(22) Filed: Mar. 25, 2009

Related U.S. Application Data
(60) Provisional application No. 61/207,860, filed on Mar. 25, 2008.

(51) Int. Cl. A61N 1/05; H05K 13/00

(52) U.S. Cl. 607/119; 29/854

(57) ABSTRACT

This disclosure relates to implantable medical devices; in particular, to medical electrical leads coupled to a conductive pressure sensor capsule and methods and apparatus for insulating the capsule with a unitary custom-molded overlay.

### CROSS-REFERENCE TO RELATED APPLICATIONS

This application claims the benefit of and priority to U.S. Provisional Application Ser. No. 61/207,860, entitled “Integrated Conductive Pressure Sensor Capsule with Custom Molded Unitary Overlay”, the contents of which are incorporated by reference herein in its entirety.

### FIELD

This disclosure relates to implantable medical devices (IMDs); in particular, to medical electrical leads coupled to a conductive pressure sensor capsule and methods and apparatus for insulating the capsule with a unitary custom-molded overlay.

### BACKGROUND

Sensors have previously been coupled to cardiac leads. Since the leads are coupled to the myocardium they must possess flexibility and strength. If one or more electrodes are disposed distal to a sensor one or more electrical conductors must pass by the sensor thereby increasing the complexity of the sensor assembly and possibly increasing the dimension of the sensor package.

Since a sensor-bearing lead typically must be fixed in place within or on the heart for consistent sensed signals, an active fixation sub-assembly is often located at the distal tip. Given the closed distal tip and active fixation a stylet is oftentimes used to extend and retract a helical shaped member before torque is applied by a torque coil to fix the helix into adjacent tissue. Thus, the torque coil is a second elongated member, optionally electrically active, that must extend beyond the sensor. In the prior art the cables and coils were simply routed around the sensor module, or package.

For a number of reasons, including the presence of electrically active tip- and ring-type electrodes located nearly, the sensor package of a physiologic sensor must be rendered electrically neutral. This has been accomplished with coating the sensor with insulating material(s) which are oftentimes of inconsistent depth and surface finish. This can also result in inconsistent material depth, air bubbles, and the like. Also, due to the thickness of the applied material the portion covering a transducer membrane, or diaphragm, such as for a capacitive pressure sensor, had to be manually removed and replaced with another insulative material (after sealing the edges where the material was removed). Besides the excess time and complexity, the possibility that the numerical yield from this type of production technique can change (i.e., whether beginning at a reasonable yield the yield can vary or drop too low to predict or to make economic sense, respectively).

A need thus exists in the art for compact physiologic sensor packaging that can easily, reliably, and efficiently be rendered electrically neutral (i.e., insulated).

### SUMMARY

Thus, herein provided are methods and structures for coupling a conductive sensor package to a distal portion of a medical electrical lead and implant the lead by temporarily inserting a stylet through a portion of the sensor package (to the distal end of the lead). Optionally one or more electrical conductors also pass through a portion of the sensor package without affecting the hermeticity thereof while providing electrical communication with one or more electrodes disposed distal to the sensor. The distal end of the lead can include an active tissue fixation member such as an extendable/retractable or fixed helical screw. Such a screw can be fixed to the distal tip of the lead, thereby requiring rotation via a stylet or of the entire lead to fixate an electrically active distal tip in a desired portion of tissue. The helical screw can be electrically active or neutral whether or not it rotates independently of the lead body or is fixed relative to the lead body. However, if electrically active redundant insulation is applied or utilized to reduce possibility of electrical short circuit or the like. Such a system can be fabricated according to the disclosure with advantages of reduced size, stability, and improved performance characteristics of a manually deployable cardiac sensing and, optionally, therapy delivery lead.

Since the conductive sensor package is typically fabricated of metal, such as titanium alloy or titanium or the like, the bores or channels can include electrical insulation intermediate each bore and/or over both the coil and cable. This insulation can be deemed redundant or fault tolerant as the coil and cable are themselves typically insulated. The insulation can include an appropriately sized polymer tube inserted into the bores or channels or placed on the coil and/or cable or a layer of material or equivalent during assembly.

One or more pacing and sensing electrodes couple to the lead distal to the sensor package. For instance, the cable can couple to a ring electrode and the torque coil can then couple to a tip-type electrode (e.g., an active fixation helix-type tip electrode). In one embodiment, a ring electrode is integrated with the sensor package, thereby reducing the length of the package. In one form of this embodiment the ring electrode resides entirely within the length of the sensor package. In another form, only a portion of the ring electrode overlies the sensor package.

A sensor capsule utilizing the present methods and apparatus can be used to sense any of a variety of physiologic parameters like pressure, acceleration and the like wherein the capsule couples to an IMD.

As noted above, electrical insulation must render the entire conductive sensor capsule electrically neutral, including the sensing membrane, if any, so that any other electrically active components implanted in a subject do not interfere with the sensor accuracy (e.g., to reduce signal artifacts) and vice versa. In addition, having a biocompatible unitary overlay reduces the chance that body fluid will corrode or invade the sensor capsule. Having an extremely consistent surface finish and thickness as provided herein also provides better accuracy and can improve the yield of an enterprise fabricating such implantable sensors.

In accordance with the foregoing, herein is provided apparatus and methods for rendering a conductive sensor package electrically neutral by fabricating a custom-molded chemically-treated biocompatible film (herein an “overlay”).

One technique involves first preparing a customized mold and related components (e.g., a suitable core pin). In one embodiment, a liquid silicone rubber (LSR) molding press is used to inject a two-part LSR into the mold having a core pin shaped identically to the outside surface of the sensor capsule—including the complex multi-surface sensing membrane depicted in the appended drawings. The LSR material is vulcanized while in the heated mold until it is cured and then removed from the core pin. The vulcanized and partially cured overlay is then post-cured to fully cure the overlay. The overlay is inspected subsequent to being fully cured and if it passes inspection any loose flash (e.g., excess material around the periphery of the overlay) not affecting the surface appearance or consistency of the overlay is removed. At final assembly, the overlay is swelled in a suitable solvent (e.g., heptane) until it is large enough to position it over the exterior of the sensor capsule, including the deflectable membrane used to sense subtle physiologic parameters. Then the overlay is allowed to dry to its original, desired dimensions. To finish the assembly a small amount of silicone medical adhesive is dispensed under the overlay around the sensor circumference and also the adjoining parts, such as a ring-type cardiac sensing and pacing electrode, and allowed to dry.

Once completely dry the sensor capsule can be joined to a suitable medical electric lead such as a defibrillation lead having one or more high voltage coil-type electrodes coupled thereto. The customized overlay thus includes the nuance of all the surface features of the sensor capsule from a unitary, consistent layer of biocompatible material. In the depicted embodiment this includes all the topography of the capsule including the multiple discrete surfaces of the sensing membrane by performing only a few simple and efficient processing steps.

The foregoing and other aspects and features will be more readily understood from the following detailed description of the embodiments thereof, when considered in conjunction with the drawings, in which like reference numerals indicate similar structures throughout the several views.

### BRIEF DESCRIPTION OF THE DRAWINGS

FIG. 1 is a perspective view of the distal portion of a pressure sensing lead body having a pressure sensor with a sensor membrane which deflects due to fluctuations in pressure in a cardiac chamber.

FIG. 2 is a cross-sectional view of a portion of a lead body wherein two major elongated lumens, a sensor lumen and a torque coil lumen are spaced apart and disposed whereby they define a plane which promotes a bending direction perpendicular to the defined plane.

FIG. 3 is a cross-sectional view of the lumens depicted in FIG. 2 and the accompanying components disposed therein; namely, a sensor bus lumen, a torque coil lumen as well as two high energy cables (SVC cable and RV coil) and a low energy pacing cable (ring cable).

FIG. 4 is an elevational side view of an exemplary sensor package illustrating an embodiment wherein a relatively thin membrane is used to sense pressure fluctuations on one side of the package and a relatively thicker back portion provides an axis of relative stiffness to the package.

FIG. 5 is a perspective view illustrating the relatively thicker back portion of the sensor wherein the back portion has two longitudinal bores for receiving an elongated conductor and a torque coil, respectively.

FIGS. 6A, 6B and 6C depict alternate view of the sensor 200 depicted in FIG. 4 and 5; namely, an elevational side view, a plan view and a cross-sectional view.

FIGS. 7A and 7B are elevational views of two related embodiments of the sensor package described and depicted herein.

FIG. 8A and 8B are perspective views of an exemplary ring-type electrode 113 used for sensing and pacing and typically disposed distal of the sensor package 200.

### DETAILED DESCRIPTION OF THE ILLUSTRATED EMBODIMENTS

In the following detailed description, references are made to illustrative embodiments for methods and apparatus including very small sensors coupled to medical electrical leads. This disclosure provides enhanced mechanical resiliency to very small sensors coupled to medical electrical leads that are cooperatively designed and fabricated.

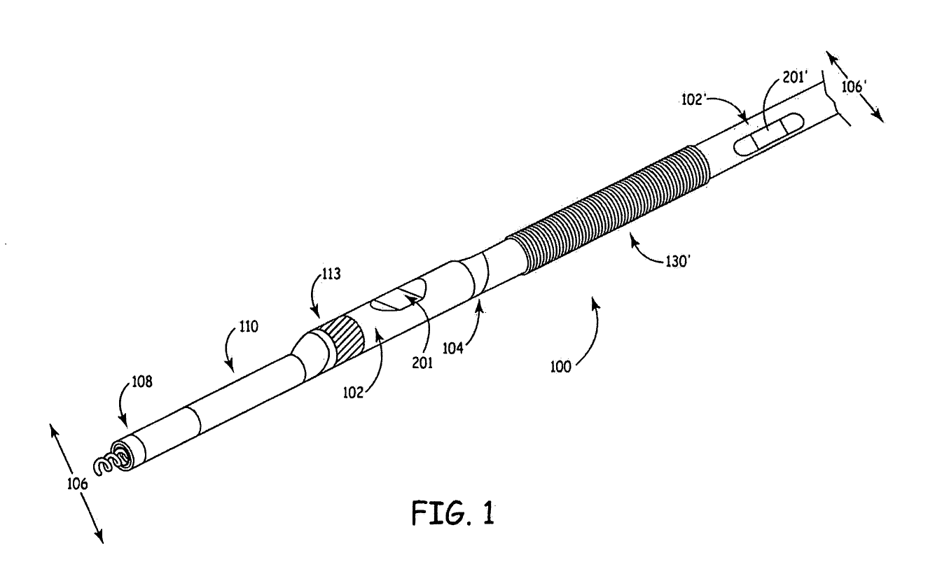

FIG. 1 is a perspective view of the distal portion of a pressure sensing lead body 100 having a pressure sensor 102 with a sensor membrane 201 which deflects due to fluctuations in pressure in a cardiac chamber. In order to best sense such fluctuations, minimize signal artifacts, and limit stress upon the sensor 102, when coupled to myocardial tissue the membrane 201 sweeps laterally (along the axis defined by arrow 106) during chronic implantation. Adjacent to the sensor 102 is optional pacing and sensing ring electrode 113. Coupled to the sensor is a relatively flexible member 110 coupling from the ring electrode 113 to optional extendable and retractable helix sub-assembly 108 used to fixate the tip of lead 100 to adjacent myocardial tissue. A proximal sensor lead portion 104 includes optional right ventricular (RV) coil electrode 130′ used for high energy defibrillation therapy delivery. Proximal to the RV coil electrode 130′ is an optional second pressure sensor 102′ having a sensing membrane 201′. Proximal of the second pressure sensor 102′ an optional superior vena cava (SVC) coil electrode (not shown) can be coupled to the lead 100.

Although not depicted in FIG. 1, within the lead body 100 in the proximal sensor lead portion 104 a set of electrical conductors reside within a multi-lumen structure. If the sensor lead 100 is designed only for sensing, two coils will extend at least to the sensor 102. The first, a torque coil, resides in a lumen and is used during implantation (to enhance the so-called “pushability” of the lead 100). The second, a co-axial communication coil resides in a different lumen for carrying signals to and from the circuitry of sensor 102. As noted above, the two coils can be used to establish a desired bending direction for the body of the lead 100 (i.e., laterally to the sensor membrane 201). This desired bending direction results from the slight compressive load placed upon the lead 100 shortly after implantation.

In other configurations, for example if the sensor lead 100 is designed for sensing pressure and cardiac activity and/or pacing a heart, then the torque coil used during implant can be electrically coupled to the tip electrode (e.g., helix of helical sub-assembly 108) and optionally another elongated cable-type conductor can be routed to the ring electrode 113. In this configuration, the desired bending direction remains the same due to the two coils orientation relative to the sensor membrane 201.

Also depicted in FIG. 1 is optional second sensor 102′ having a sensor membrane 201′ which can have an arbitrary orientation relative to sensor member 201 applying the principles described and depicted herein. That is, in the event that the second sensor 102′ is intended to sense pressure within the right atrium (RA) the relative orientation of the two sensors 102,102′ can be different or changed during fabrication of the lead 100 to promote a different lateral motion for the sensor 102′ (as depicted by arrow 106′). If the second sensor 102′ is adapted to sense RA pressures then beside having lateral motion of the membrane 201′ relative to the lead 100, the membrane 201′ should face away from the nearest wall of the RA. Also, the second sensor 102′ can utilize the same digital sensor protocol carried upon the sensor communication bus as the first sensor 102.

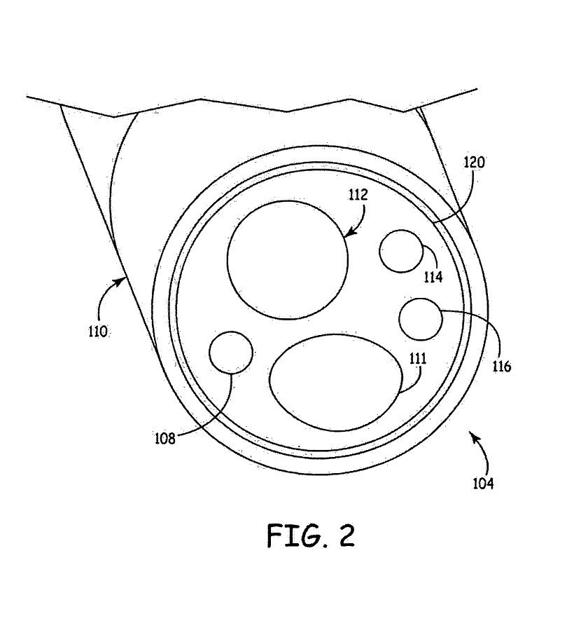

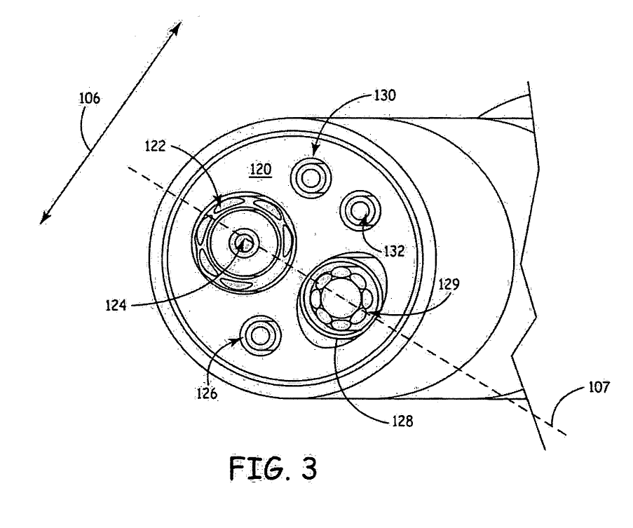

FIG. 2 is a cross-sectional view of a portion of a lead body 104 wherein two major elongated lumens 111,112 (denoted as a sensor bus lumen and a torque coil lumen) are spaced apart and disposed whereby they define a plane through the center axis of each which promotes a desired bending direction perpendicular to the defined plane. As depicted the lead body portion 104 also has three other smaller-diameter lumens 108,114,116 configured to receive an SVC cable, an RV cable, and a ring electrode cable lumen, respectively. The lead body 104 is sheathed in an overlay tubing 110 and the penta-lumen 120 is nominally fabricated of Silicone (e.g., MED-4755 made by Nusil Technology of Carpinteria, Calif.). As depicted the major lumens 111,112 are designed to promote the desired bending direction (indicated generally by arrow 106 of FIG. 3).

FIG. 3 is a cross-sectional view of the lumens depicted in FIG. 2 and the accompanying components disposed therein; namely, an inner sensor bus cable 124 and an outer sensor bus coil 122, a torque coil 129 having an optional covering 128, as well as two high energy cables (SVC cable 126 and RV cable 130) and a low energy pacing and sensing cable (ring cable) 132. The sensor bus coil 122, the sensor bus cable 14, and the torque coil 129 define a plane through the axial center of each (depicted by dashed line 107) and the desired bending direction lies generally perpendicular to this plane (106 in FIG. 3).

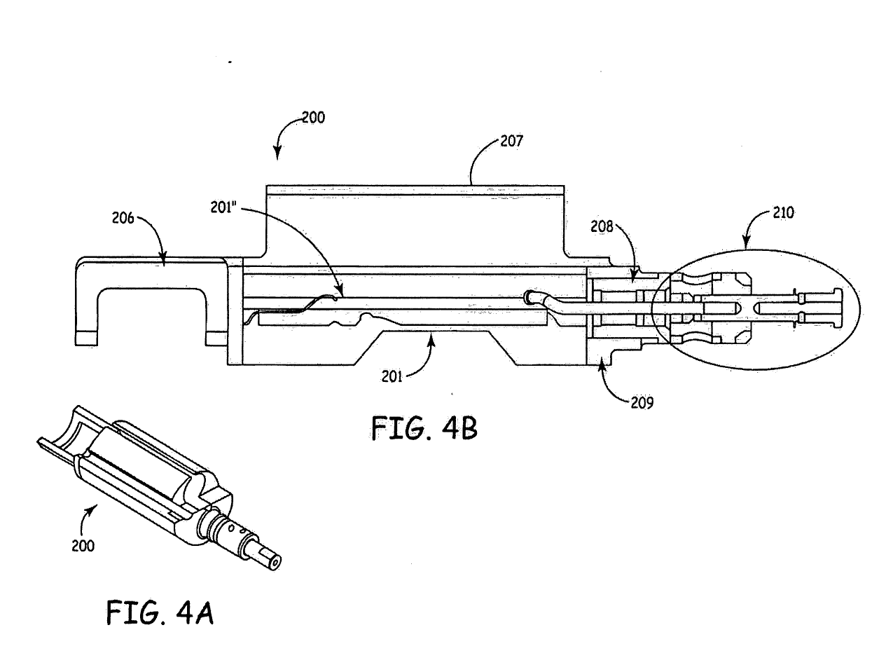

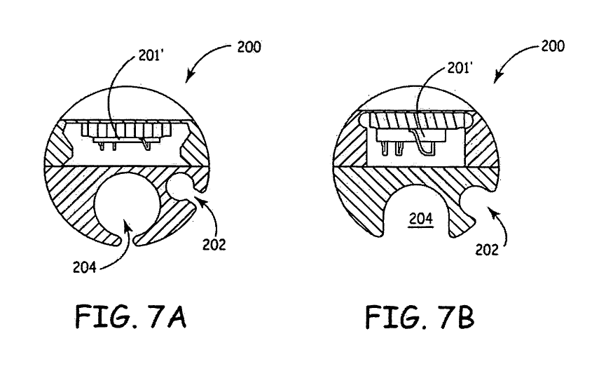

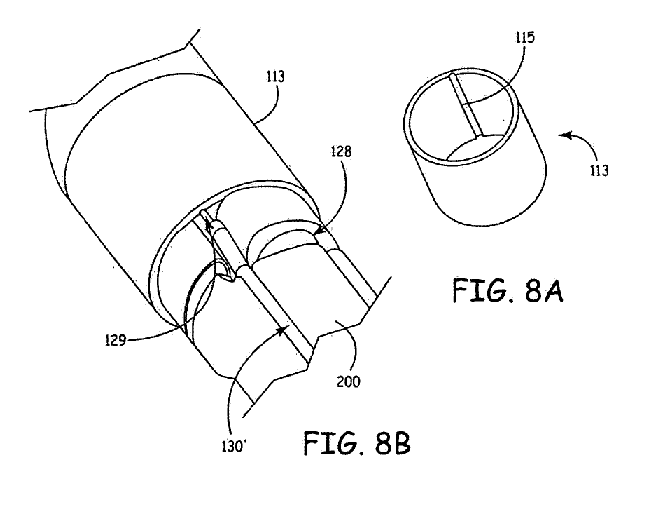

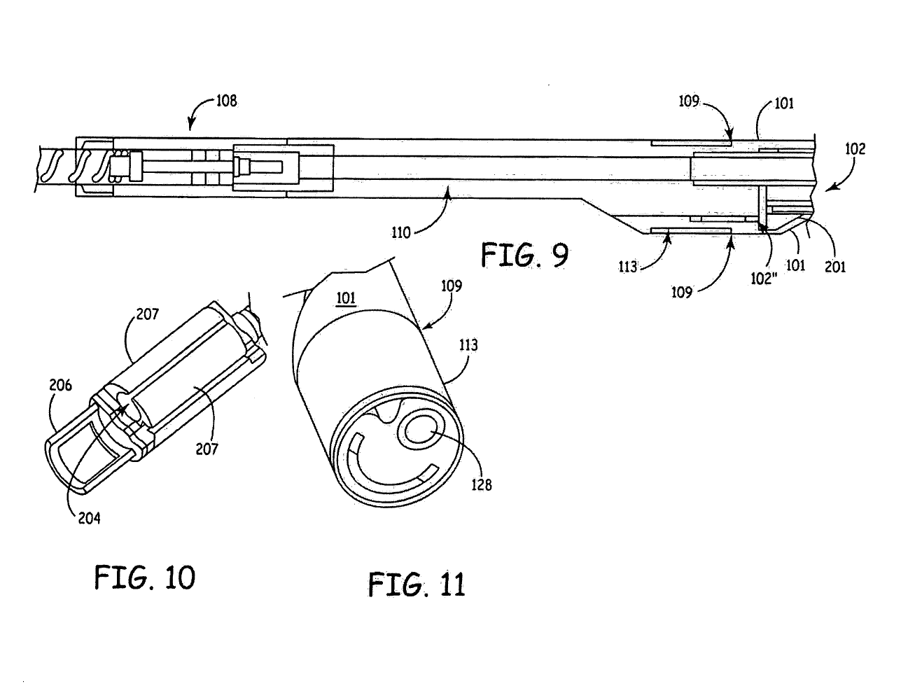

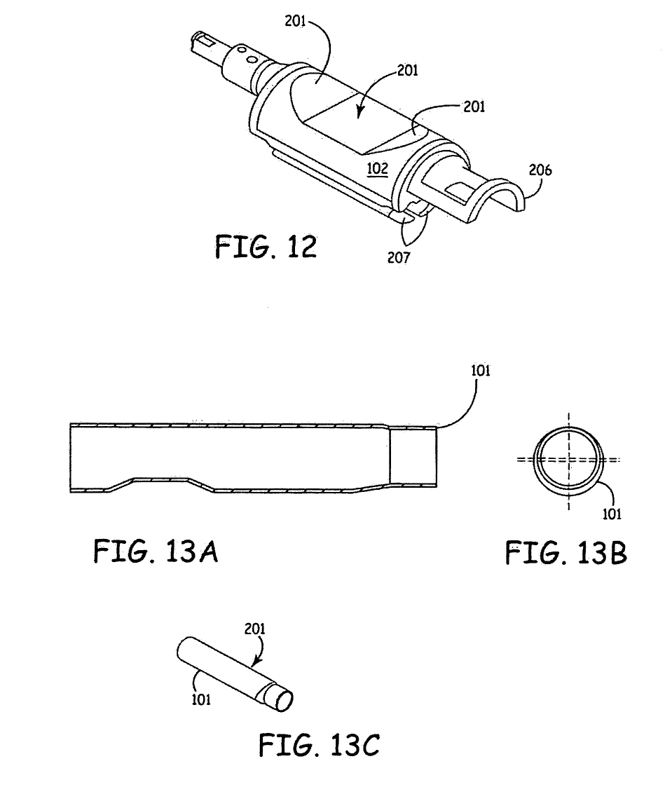

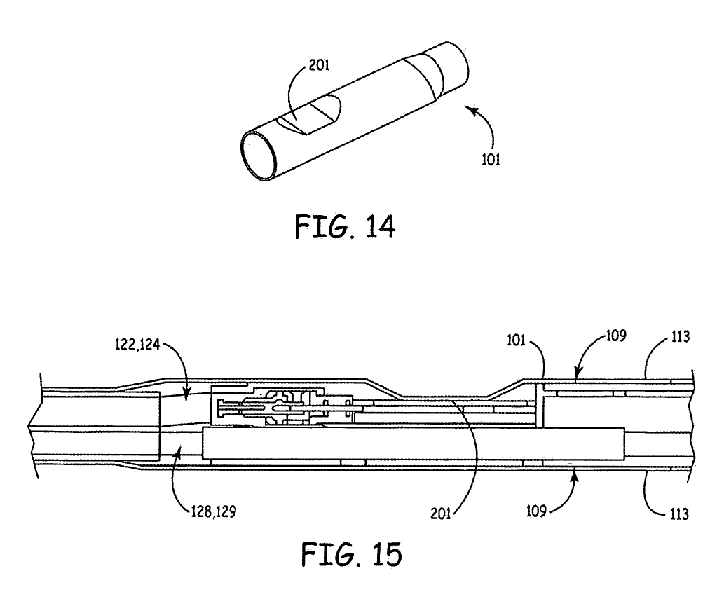

FIGS. 4A and 4B depict an embodiment of a sensor package 200 designed and constructed out of titanium according to one form of the invention. For example, a suitable titanium alloy includes Ti 6Al-4V although other alloys and other materials could suffice. FIG. 4A is a perspective view of the package 200 and FIG. 4B is an elevational side view of the sensor package 200 illustrating an embodiment wherein a relatively thin membrane 201 is used to sense pressure fluctuations on one side of the package 200 and a relatively thicker back housing portion 207 provides an axis of relative stiffness to the package 200 (which is generally perpendicular to the package 200 depicted in FIG. 4B (i.e., perpendicular to the drawing sheet). In practice the axis of stiffness is designed so that it is aligned with the desired bending direction 106,106′ of the lead body 104 that is provided by the twin coils described above (and other structures and/or lumens described below in relation to FIGS. 7-10). A distal adapter 206 can is integrated to the sensor package and flexible distal end portion 110 (depicted in FIG. 1) which provides incremental desired bending direction due to the torque coil therein and the proximity to both the rigid sensor package 200 (including distal adapter 206) and the dual-coil proximal lead portion 104. The distal adapter increases the stiffness of the overall package that adds signal accuracy to the output signal. The distal adapter also adds functional attachment, or anchoring structure, for example, if a ring electrode (see FIG. 8A and 8B) are wholly or partially disposed over the sensor package (including adapter portion 206). An advantage to a ring electrode wholly overlying the adapter portion of the package 200 is that the length of the sensor package can be reduced. An integrated circuit 201″ adapted to at least of one of convey signals and calculate pressure applied to the membrane 201. The lead adapter 209 is designed to maintain alignment between the desired bending direction of the lead body and the axis of relative stiffness of the package.

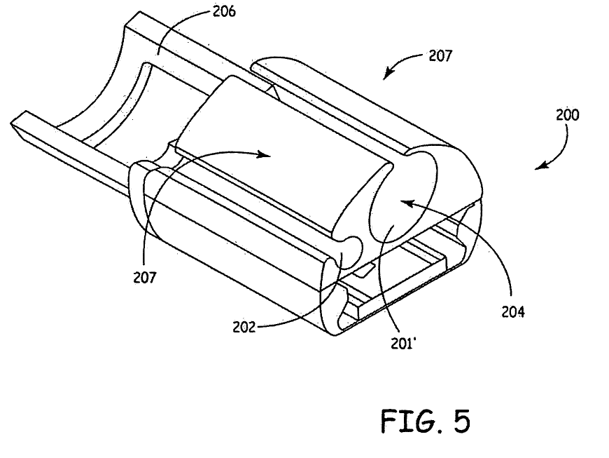

FIG. 5 is a perspective view illustrating the relatively thicker back housing portion 207 of the sensor package 200 wherein the back housing portion 207 has two longitudinal bores 202,204 for receiving an elongated conductor to coupled to a distal ring electrode and a torque coil, respectively (not shown in FIG. 5). The bores 202,204 are depicted having an open longitudinal portion but such a portion is not required to practice the foregoiong. In fact, the collar of the open portion of bores 202,204 can extend radially outward from a position approximately from the maximum diameter of each respective bore. A portion of the pressure sensor integrated circuit 201″ is also depicted in FIG. 5 disposed within the package 200.

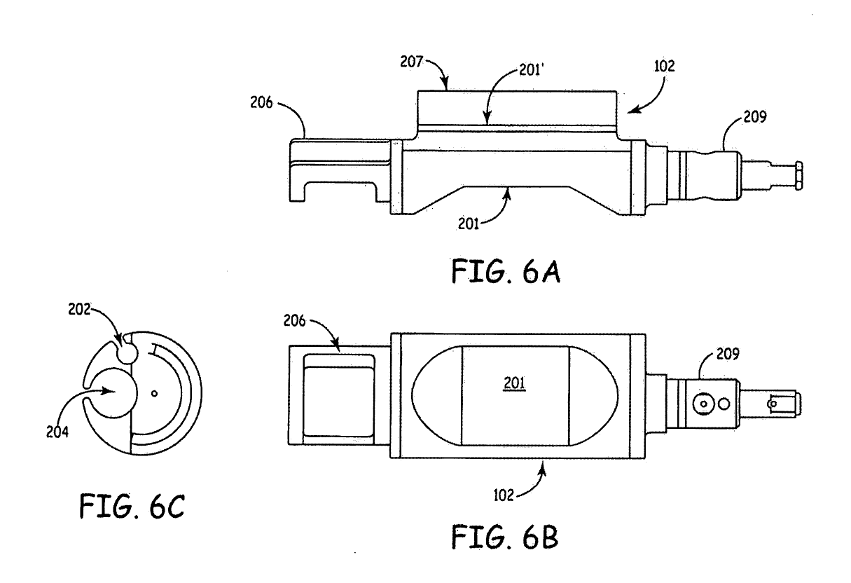

FIGS. 6A, 6B and 6C depict alternate views of the sensor package 200 depicted in FIG. 4 and 5; namely, an elevational side view, a plan view and a cross-sectional view. The bores 202,204 of relatively thicker back portion 207 and the generally circular cross-sectional shape of the sensor 102 are depicted in FIG. 6C. The proximal and distal adapter 209,206 are also depicted. Whether or not the distal adapter 206 is bonded, seam welded (with a laser welder) or milled from a unitary portion of conductive material, it is considered to be part of the overall sensor package 200.

FIGS. 7A and 7B are elevational views of two related embodiments of the sensor package described and depicted hereinabove. In essence the two depicted structures are very similar but nevertheless illustrate that besides one or both bores 202,204 being completely closed (as shown in FIG. 6C), one or both can be partially open (FIG. 7A) or substantially open (FIG. 7B). Also shown in FIGS. 7A and 7B, is the interior hermetic portion wherein the sensing circuitry 201′ and sensor are coupled to the interior of the sensing membrane. Also illustrated is the fact that at least part of the sensor package 200 has a substantially circular cross section (e.g., at least the opposing end portions). Such a cross section, even if just partial, improves the ease and desirability of implanting such medical electrical leads by reducing changes in the overall diameter and shape of the lead.

FIG. 8A and 8B are perspective views of an exemplary ring-type electrode 113 used for sensing and pacing and typically disposed distal of the sensor package 200. As shown in FIG. 8A, the interior of the ring electrode 113 has a groove 115 for receiving the distal end portion of the cable conductor 129. As depicted the ring electrode 113 resides on an electrically insulative flexible distal tip portion of the lead. However, assuming adequate electrical insulation disposed between the metallic sensor package 200 and the ring electrode 113, the ring electrode 113 could safely reside wholly, or partially, over a part of the sensor package 200. In a related aspect (and as depicted in FIG. 8B), the cable conductor if covered in insulation 130′ and the torque coil is also covered with insulation 128. The latest embodiment have the advantage of further reducing the overall size of the sensor package, among other advantages.

FIG. 9 is an elevational view in cross section of the distal portion of the lead having a helical screw tip electrode 108 a flexible distal portion 110 coupled to the sensor 102. The distal portion serves as both a tip electrode to ring electrode spacer and provides flexibility and dampened motion for the sensor 102 once implanted. A ring electrode 113 couples to the sensor capsule 102 via the distal adapter portion shown in FIG. 10) and adjacent the customized unitary overlay 101. During fabrication, medical grade adhesive is dispensed circumferentially in the seam 109 between the ring electrode 113 and the overlay 101.

The silicone sensor overlay 101 electrically isolates the sensor capsule 200 from the electrodes 108,113,130′ of the lead body 100 and provides a uniform layer of insulation over the sensor diaphragm 201 in order to maintain a consistent interface between body fluid and the sensor capsule 200 since motion of the diaphragm 201 is translated into pressure difference. The overlay 101 is also necessary to prevent any artifacts from pacing pulses from interfering with the pressure signal. In one embodiment (not having a ring electrode distal immediately distal to the sensor capsule 200), the overlay 101 is bonded with suitable medical adhesive (at periphery 109) to the flexible distal portion 110 (used as a tip-to-ring spacer) at one side of the capsule 200 and the proximal lead body portion 104 at the other side providing strength and sealing of the capsule 200. The inside surfaces of the overlay 101 are the same shape as the capsule 200 providing a conformal fit and, when backfilled with silicone medical adhesive, provides adhesion and intimate contact between the sensor capsule 200 and the overlay 101 allowing the overlay 101 to move in union with the sensor diaphragm. In one embodiment employing a pressure sensor as depicted herein, the overlay is on the order of 0.004 to 0.006 in. thickness.

Now referring to FIGS. 10 and 11, which are perspective views of the distal end portion of the sensor capsule 200, the distal 206 adapter portion of the sensor capsule 200 is depicted without and with a ring electrode 113, medical adhesive seam 109, and overlay 101, respectively. The elongated lumen 128 for the torque coil 128 (not depicted in FIGS. 10 or 11) is also shown in FIG. 11 as is the bore 204 for the torque lumen in FIG. 10.

FIG. 12 is a perspective of the sensor capsule 200 and the proximal adapter 209 of a commercial embodiment illustrating nominal membrane dimensions and a nominal thickness of the sensing membrane. Also depicted is the back housing 207 and distal adapter 206 portion of the capsule 200.

FIGS. 13A-C are cross-sectional, plan, and perspective views, respectively, of an overlay 101 as taught, described, and depicted herein. The overlay 101 is molded in a liquid silicone rubber (LSR) molding press by injecting a two-part LSR fluid into a mold whose core pin is shaped identically to the outside surface of the sensor capsule 200 including the complex recessed diaphragm 201 portion of the capsule. The injected rubber is vulcanized in the heated mold until it is at least partially cured and then removed from the core pin. The overlay 101 is then post-cured to a fully cured state and inspected and any loose flash removed. At final assembly the overlay 101 swells in fluid contact with a suitable solvent (e.g., heptane) until it is large enough to position the overlay 101 over an assembled sensor capsule 200. The overlay 101 is allowed to dry and shrink to its original size and shape and then a small amount of silicone medical adhesive is dispensed under the overlay 101 around the circumference of the sensor capsule 200 and also to the adjoining parts (e.g., ring electrode, proximal lead body portion, or distal portion) and the adhesive is allowed to dry. This design and method of manufacture saves significant amount of time and cost versus previous methods of coating a conductive sensor package and also offers acceptable pressure sensing performance.

It will be understood that specifically described structures, functions and operations set forth in the above-referenced patents can be practiced in conjunction with the present invention, but they are not essential to its practice. It is therefore to be understood, that within the scope of the appended claims, the invention may be practiced otherwise than as specifically described without actually departing from the spirit and scope of the present invention. For example, the sensor could comprise an accelerometer (single- or multi-axis) which for any of a number of reasons might need to have reduced structure on one or more sides thereof thus becoming susceptible to the objects solved herein.

1. A medical electrical lead, comprising:
an elongated lead body formed of a biocompatible material;
a conductive hermetic sensor package coupled to the lead body; and
a unitary conformal non-conductive overlay film surrounding substantially the entire exterior of the sensor package.

2. A lead according to claim 1, wherein sensor package includes a deflectable region relative to another portion of the sensor package.

3. A lead according to claim 2, wherein the deflectable region comprises a recessed region.

4. A lead according to claim 1, wherein the deflectable region comprises a region having at least two portions disposed at an angle relative to each other.

5. A lead according to claim 2, further comprising one of a pressure sensor and an accelerometer coupled to the deflectable region.

6. A lead according to claim 1, wherein the conductive sensor package is fabricated of one of a titanium alloy and titanium.

7. A lead according to claim 1, further comprising a ring-type electrode coupled next to the distal edge of the overlay film.

8. A lead according to claim 7, further comprising a volume of medical grade adhesive disposed between the proximal edge of the ring-type electrode and the overlay film.

9. A lead according to claim 1, further comprising a cylindrically-shaped member coupled next to the distal edge of the overlay film.

10. A lead according to claim 7, further comprising a volume of medical grade adhesive disposed between the proximal edge of the cylindrically-shaped member and the distal edge of the overlay film.

11. A lead according to claim 1, further comprising a cylindrically-shaped member coupled next to the proximal edge of the distal edge of the overlay film.

12. A lead according to claim 7, further comprising a volume of medical grade adhesive disposed between the distal edge of the cylindrically-shaped member and the proximal edge of the overlay film.

13. A lead according to claim 1, wherein opposing end portions of the sensor package have a substantially circular axial cross-section.

14. A lead according to claim 1, wherein the overlay film comprises silicone.

15. A medical electrical lead, comprising:
an elongated lead body formed of a biocompatible;
a conductive sensor package coupled to the lead body; and
a unitary conformal non-conductive overlay film surrounding the entire exterior surface of the sensor package.

16. A lead according to claim 15, wherein the deflectable member comprises one of a deflectable membrane, a deflectable diaphragm, an accelerometer.

17. A lead according to claim 15, wherein the sensor package includes a distal adapter member and further comprising a ring-type electrode one of wholly and partially overlying the distal adapter member.

18. A lead according to claim 17, further comprising:
a customized, unitary silicone overlay disposed over the entire exterior surface of the sensor package.

19. A method of fabricating a medical electrical lead, comprising:
providing a conductive sensor capsule, wherein said sensor capsule has at least one exposed deflectable region and a particular surface topography;
inserting the sensor capsule into a swollen custom molded silicone vessel that has an interior surface that corresponds to the particular surface topography; and
allowing the capsule and the silicone vessel to dry until the interior surface of the silicone vessel closely conforms to the particular topography.

20. A method according to claim 19, further comprising:
coupling a proximal end of the sensor package to an elongated medical electrical lead body.

21. A method according to claim 19, wherein the silicone vessel was swollen due to contact with a solvent.

22. A method according to claim 20, wherein the solvent comprises heptane or an isomer of heptane.

23. A method according to claim 20, wherein the contact comprises one of immersion, sputtered, sprayed.

24. A method according to claim 15, wherein the capsule comprises one of a titanium alloy and titanium.

25. A method according to claim 15, further comprising:
applying medical grade adhesive sufficient to seal the edges of opposing ends of the sensor capsule and the silicone vessel together.

26. A method according to claim 15, wherein the particular topography includes a recessed region.
